(19) United States
(12) Patent Application Publication
(10) Pub. No.: US 2021/0379543 A1
(43) Pub. Date: Dec. 9, 2021

(54) PROCESS FOR PRODUCING A NANO-TAN IIA MICROEMULSION SYSTEM

(71) Applicant: Wakamono Corporation, Ho Chi Minh City (VN)

(72) Inventors: Hong Ngoc Thi Dan, Ho Chi Minh City (VN); Nam Hai Lai, Ho Chi Minh City (VN)

(73) Assignee: Wakamono Corporation, Ho Chi Minh City (VN)

(21) Appl. No.: 17/410,961

(22) Filed: Aug. 24, 2021

(51) Int. Cl. B01F 3/08; A61K 9/107

(52) U.S. Cl. CPC B01F 3/0815; B01F 3/0811; A61K 9/1075; B01F 3/2261; B01F 2003/0834; B01F 2003/0842; B01F 2003/0896

(57) ABSTRACT

The present invention relates to a process of producing a nano-Tan IIA microemulsion system comprising the following steps: (i) preparing a dispersed phase by dissolving Tan IIA in ethanol solvent in a ratio of mass of Tan IIA:volume of ethanol solvent of 8:10; (ii) preparing a carrier by heating liquid PEG (polyethylene glycol) to 60-80° C.; (iii) adding the carrier to the dispersed phase in a mass ratio of 40:60 with further heating of the dispersed phase to 40-60° C.; (iv) elmusifying by heating until the temperature reaches 100° C., adding ACRYSOL K-140 to the mixture of the carrier and dispersed phase obtained in step (iii) in a mass ratio of 40:60 with further stirring at 500-700 rpm at about 100° C. under vacuum; and (v) filtering the product by injection through a nanofilter system before filling-packing.

### BACKGROUND OF THE INVENTION

Tan IIA, or Tanshinone IIA, is extracted from the roots of danshen or Salvia miltiorrhiza Bunge of the Labiatae family. Danshen has been widely used in Asian countries, particularly China, to treat different circulatory disorders due to its special pharmacological effects, including vasodilation, anticoagulation, anti-inflammation, and reduction of free radicals. Tan IIA helps adjust or prevent the metastases of cancer cells by adjusting the adhesion molecules. In addition, other studies have proved that Tan IIA is capable of strong anti-inflammation and anti-oxidation. However, Tan IIA has low bioavailability, which via oral administration has been proven to affect the clinical applicability of the compound.

Therefore, it is desirable to improve the absorbability and increase the bioavailability of the compound. The application of nanotechnology is a new technological application to produce a drug delivery system and increase the bioavailability of a compound. With a particle size below 100 nm, the absorption and retention capacity is increased. Tan IIA is packaged in the nano drug delivery system to help selectively, effectively, and economically deliver the compound to its targets. Nanotechnology is still new in biomedicine, and has attracted a lot of research interests. Currently, the most popular studies are about the application of nano curcumin and the drug delivery systems to targeted cells, but there has not been any study on the production of nano Tan IIA known to the present inventor. The use of a nanoparticle-forming liposome system to carry drug and release drug is a new direction to treat diseases, especially epilepsy and cancer in the future.

LIU JIANPING et al., in Chinese Patent Publication No. CN1215839C, related to a method of preparing tanshinone solid lipid nanoparticles, which, in an inventive process, produced an average diameter of tanshinone solid lipid nanoparticles of 119.7 nm, with 95% of the particle sizes fell below 130 nm. With this invention, the generated particles are still larger than 100 nm, and the encapsulation efficiency is only 81.60%.

XINGMU GUO et al., in Chinese Patent Publication No. CN102688151B, related to a method of preparing a microemulsion system of tanshinone, which, according to an inventive process, produced a particle size of less than 100 nm. However, due to the complexity of the process, it cannot be applied in industrial manufacturing.

Anitha Krishnan Nair et al., in US Patent Publication No. 2011/0229532 A1, related to a process for producing a microemulsion system of compounds belonging to an oleophilic polyphenol group by using ultrasounds with non-ionic surfactants and a non-ionic solvent to enhance water solubility. In particular, the invention related to a process for nanoization of curcumin and its derivatives, which is non-applicable to Tan IIA with uneven particle size.

Therefore, there is a demand of a process for producing a microemulsion system comprising uniform micelles of a size smaller than 100 nm with better water-solubility while retaining the structure, and activity of Tan IIA during nanoization.

### SUMMARY OF THE PRESENT INVENTION

The present invention is a process for producing a nano-Tan IIA microemulsion system that produces uniform particles of a size smaller than 100 nm with water solubility, whose unchanged activity and structure increases the use efficiency of Tan IIA active agents, in particular, in asorbability and in bioavailability. The process for producing a nano-Tan IIA microemulsion system of the present invention comprises the following steps:

(i) preparing a dispersed phase by dissolving Tan IIA in ethanol solvent in a ratio of the mass of Tan IIA to the volume of ethanol solvent of 8:10 by a stirrer at 300-500 rpm while heating to 40-60° C. for 4-8 hours;

(ii) preparing a carrier by heating liquid PEG (polyethylene glycol) to 60-80° C. with constant stirring;

(iii) adding the carrier to the dispersed phase in a mass ratio of 40:60 with further heating of the mixture of the carrier and the dispersed phase to 40-60° C. while stirring at 400-800 rpm;

(iv) elmusifying by: heating the mixture of the carrier and the dispersed phase obtained in step (iii) until the temperature reaches 100° C., adding ACRYSOL K-140 to the mixture of carrier and dispersed phase in step (iii) in a mass ratio of 40:60 with further stirring at 500-700 rpm at about 100° C. under vacuum, wherein the reaction temperature is kept at 100° C. for 3-5 hours, controlling the quality of the resulting product by water dissolution and transparency measurement, where if transparency is not reached then continue heating and measuring transparency every 30 minutes until transparancy is observed to quench the reation, with the temperature decreased for 30-60 minutes to 40-60° C.; emulsifying the entire mixture for 30 minutes at 400-800 rpm; and

(v) filtering the product by injection through a nanofilter system before filling-packing.

### BRIEF DESCRIPTION OF THE DRAWINGS

FIG. 1 is a photograph comparing the water-dispersability between a known 90% Tan IIA and a nano-Tan IIA obtained by a process for producing a nano-Tan IIA microemulsion system of the present invention; and

FIG. 2 is a graph of a SEM spectra of the size of Tan IIA nanoparticles obtained by a process for producing a nano-Tan IIA microemulsion system of the present invention.

### DETAILED DESCRIPTION OF THE INVENTION

A process for producing a nano-Tan IIA microemulsion system of the present invention is performed according to the following steps.

(i) First step: preparing a dispersed phase by dissolving Tan IIA in ethanol solvent in a ratio of the mass of Tan IIA to the volume of ethanol solvent of 8:10 by a stirrer at 300-500 rpm in combination with heating at 40-60° C. for 4-8 hours. The inventors used ethanol as a solvent that is capable of dissolving Tan IIA well, which helps form a better dispersed phase and facilitate the combination of the dispersed phase with a PEG carrier. The use of hydroxyl-(OH—) based ethanol solvent forms linkage with water, thus having a stablizing effect on the structure of the oil-in-water microemulsion system. By experiments, the inventors have detemined that, in a Tan IIA:ethanol (mass:volume) ratio of 8:10, Tan IIA achieved the highest solubility and avoided excess ethanol solvent that causes waste. The use of stirring and heating to produce Tan IIA with better dispersibility, when the inventors tested under various stirring and temperature conditions, shows that at 300-500 rpm in combination with heating at 40-60° C., the Tan IIA dispersed phase is better and combines with the PEG carrier better.

(ii) Second step: preparing a carrier by heating liquid PEG (polyethylene glycol) to 60-80° C. with constant stirring.

When used, Tan IIA is often degraded in the digestive tract, a portion of which absorbed into the blood, while the rest mostly subjected to clearance. Thus, there is a need for a process for producing micelles containing Tan IIA active agents of small size, biofilm, stable structure, non-aggregation, and high solubility. Because the microemulsion system of the present invention is employed in food and pharmaceutical industries, the agents selected for use must have great safety, no toxicity, and less side effects.

Many studies have indicated that drug deliveries may be improved in effectiveness by vehicle systems derived from a variety of polymers, and processes of using a PEG (Polyethylene Glycol) carrier which is a long chain polymer with a general formula of HO(CH2CH2)nH. Polymeric carriers with relatively high drug loads may confer many pharmacokinetic benefits, such as stablized drugs, which may be administered for treatment over long term by slow drug release in accordace with polymer decomposition, biological distribution of the drugs, targeting ability, penetration through cell membranes, etc., that can be controlled by physicochemical properties of the polymers.

(iii) Third step: adding the carrier to the dispersed phase in a mass ratio of 40:60, with further heating of the mixture of the carrier and the dispersed phase to 40-60° C. while stirring at 400-800 rpm.

(iv) Fourth step: elmusifying by heating the mixture of the carrier and the dispersed phase obtained in the third step (iii) until the temperature reaches 100° C., adding ACRYSOL K-140 to the mixture of carrier and dispersed phase in the third step in a mass ratio of 40:60 with further stirring at 500-700 rpm at about 100° C. under vacuum, wherein the reaction temperature is kept at 100° C. for 3-5 hours, controlling the quality of the resulting product by water dissolution and transparency measurement, where if transparency is not reached then continue heating and measuring transparency every 30 minutes until transparancy is observed to quench the reation, with the temperature decreased for 30-60 minutes to 40-60° C. Next, emulsify the entire mixture for 30 minutes at 400-800 rpm.

By theoretical and experimental studies, the inventors found that to produce nano-Tan IIA with good water solubility, the emulsion system should be in the form of an oil-in-water emulsion. Emulsifier selection to enhance the stability of the microemulsion system was based on the propeties thereof (in the form of oil-in-water microemulsion system, in the form of water-in-oil microemulsion system, etc.) Thus, the inventors selected ACRYSOL K-140 as the emulsifier, because ACRYSOL K-140 is a hydrophilic, non-toxic, and highly safe agent. The inventors had to carry out multiple studies to determine that when the PEG:ACRYSOL K-140 ratio is 40:60 by mass, it may generate stable polymer chains.

As the emulsifier ACRYSOL K-140 is a molecule with 2 distinct portions, an oleophilic portion and a hydrophilic portion, it is capable of forming a linkage with Tan IIA and the carrier mixture. The oleophilic portion of ACRYSOL K-140 forms a linkage with Tan IIA and the hydrophilic portion of ACRYSOL K-140 forms a linkage with the hydrophilic portion of the mixture of the PEG carrier, thus forming nano-Tan IIA micelles and with which stucture maintaining good protection of Tan IIA.

A nano-Tan IIA microemulsion system is produced by stirring at 500-700 rpm under vacuum, wherein the reaction temperature is kept at 100° C. for 3-5 hours, then emulsification of the entire mixture for 30 minutes at 400-800 rpm.

The microemulsion system obtained by a process of the present invention has a pH of 7-7.4. With this pH, the micelles are stable since the linkage between the Tan IIA and the carrier material is kept in dispersion in this neutral environment, while the microemulsion system has pH<7 then this linkage weakens resulting in degradation of the Tan IIA nanoparticles in the digestive tract.

The nano-Tan IIA microemulsion system obtained by the process of the present invention with HLB (hydrophilic lipophilic balance of 0-40) of 13-18 is a hydrophilic microemulsion system. The microemulsion system comprises non-aggreagated hydrophilic micelles containing Tan IIA with a particle size of 20-80 nm, so it may easily permeate across cell membranes to take effect and increase the solubility of Tan IIA in water, thereby enhancing the bioavailability thereof.

(v) Fifth step: filtering the product by injection through a nanofilter system before filling-packing to remove excess agents and ensure solution uniformity and stability.

### EXAMPLES

### Example 1: Production of 58 g of Nano-Tan IIA Microemulsion System

A dispersed phase was prepared by dissolving 8 g of Tan IIA in 10 mL of 96% ethanol with a stirrer at 400 rpmin combination with heating to 40° C. for 6 hours to form a homogeneous solution to form by 1000 W IKA C-MAG HS 7 magnetic hotplate stirrer. 15 g of the dispersed phase obtained (ethanol 96% partially evaporated during heating stirring).

A carrier is prepared by heating 10 g of PEG to 60° C. with constant stirring.

10 g of the carrier was added to 15 g of the above-prepared dispersed phase (the carrier:dispersed phase ratio is 40:60 by mass), this mixture of the carrier and the dispersed phase continued to be heating to 60° C. and stirred at 600 rpm using a 1000 W IKA C-MAG HS 7 magnetic hotplate stirrer. 35 g of the mixture of the carrier and the dispersed phase was obtained.

Then, 35 g of the obtained mixture of the carrier and the dispersed phase was heated until the temperature reached 100° C., 23.3 g of ACRYSOL K-140 was added to 35 g of the mixture of the carrier and the dispersed phase obtained above with further stirring at 700 rpm under vacuum, wherein the reaction temperature was kept at 100° C. for 5 hours. The quality of the resulting product was controlled by water dissolution and transparency measurement, where if transparency is not reached then the heating and transparency measurement would continue every 30 minutes until transparancy was observed to quench the reation, with the temperature is decreased for 30-60 minutes to 50° C.; the entire mixture emulsifyed for 30 minutes at 800 rpm using an 800 W IKA T25 DIGITAL ULTRA-TURRAX homogenizer.

Before filling, the product was injected via a nanofilter system with a purpose of removing excess Tan IIA which did not form micelles, to give a nano-Tan IIA microemulsion system which dispersed well in water.

By UV-vis spectroscopies, the inventors found that the peak positions of the Tan IIA ingredient and the nano-Tan IIA microemulsion system were completely matching. This showed that the microemulsion system obtained by the process of the present invention still retained Tan IIA structure and activity during nanoization. UV-vis spectrocopies were used to quantify Tan IIA content in the microemulsion system. Results showed that the concentration of Tan IIA in the nano-Tan IIA microemulsion system was about 10%.

Size measurement of Tan IIA nanoparticles was conducted by a scanning electron microscope SEM, showing that the particle size was in the range of 20-80 nm.

SEM, or Scanning Electron Microscope, is a type of electron microscope that is capable of producing high-resolution images of the surface of a specimen using a narrow electron beam (a beam of electrons) to scan over the specimen surface. The imaging of the specimen is performed through the recording and analysis of the radiation emitted from the interaction between the electron beam and the specimen surface.

TABLE I

| Size | Size | Zeta | | |
|---|---|---|---|---|
| (nm, according | (nm, according | potential | Stability | Water |
| to SEM) | to DLS) | (mV) | (month(s)) | solubility |
| 20-80 | 20-80 | −40 | >12 | Good water |
| | | | | solubility, after |
| | | | | dissolution in |
| | | | | water, the system |
| | | | | stabilized >7 days |

The above results showed that the use of the PEG carrier with ACRYSOL K-140 gave a microemulsion system comprising micelles of small size (20-80 nm), high stability (>12 months), good water solubility and after dissolution in water, the system stabilized >7 days.

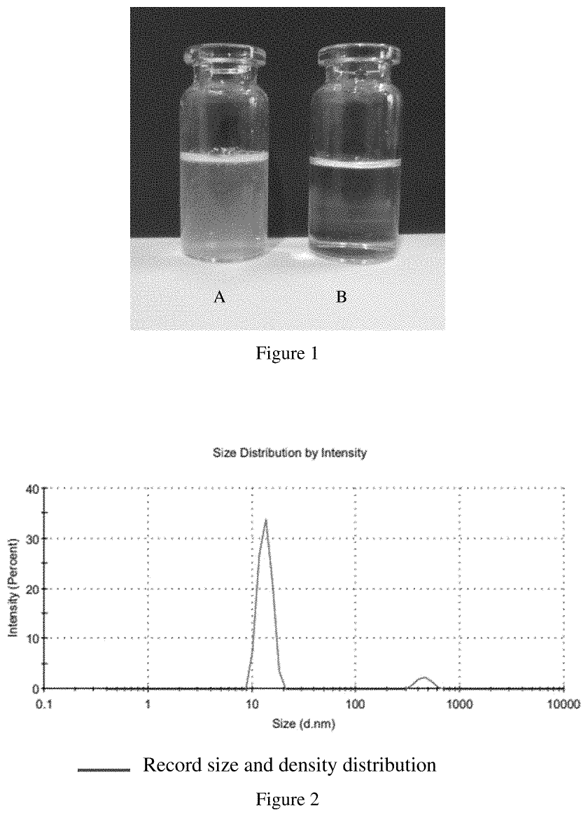

FIG. 1 is a picture comparing the water-dispersability between known 90% Tan IIA and nano-Tan IIA obtained by a process of the present invention, in which bottle A showed the known 90% Tan IIA dispersed in water, and bottle B showed the nano-Tan IIA dispersed in water obtained by the process of the present invention. The photograph shows that the known 90% Tan IIA was insoluble in water, formed suspending particles in water, that the solution was slurry, deposited at the bottom of the bottle over time. Conversely, the nano-Tan IIA obtained by the process of the present invention was completely dispersed in water, forming a transparent and homogeneous solution.

FIG. 2 shows the SEM spectrum of the size of Tan IIA nanoparticles obtained by the process of the present invention. It was found that the particles were uniform in size, in the range of 20-80 nm, with high density of 100%.

### Advantageous Effects of the Invention

The process for producing a nano-Tan IIA microemulsion system of the present invention has succeeded in producing a microemulsion system comprising uniform nano-Tan IIA micelles of small size, in the range of 20-80 nm, with good solubility in water while retaining Tan IIA structure and activity during nanoization.

The agents used in production of nano-Tan IIA, which are dispersed well in water, are highly safe and non-toxic, and have fewer side effects, so the nano-Tan IIA microemulsion system obtained by the process of the present invention has great safety when used.

The process of the present invention is simple, easy to perform, and suitable for current practice.

We claim:

1. A process for producing a nano-Tan IIA microemulsion system, comprising:
(i) preparing a dispersed phase by dissolving Tan IIA in ethanol solvent in a ratio of mass of Tan IIA:volume of ethanol solvent of 8:10 by a stirrer at 300-500 rpm while heating to 40-60° C. for 4-8 hours;
(ii) preparing a carrier by heating liquid PEG (polyethylene glycol) to 60-80° C. with constant stirring;
(iii) adding the carrier to the dispersed phase in a mass ratio of 40:60 with further heating of the mixture of the carrier and the dispersed phase to 40-60° C. and stirring at 400-800 rpm;
(iv) elmusifying by heating the mixture of the carrier and the dispersed phase obtained in step (iii) until the temperature reaches 100° C., adding ACRYSOL K-140 to the mixture of carrier and dispersed phase in step (iii) in a mass ratio of 40:60 with further stirring at 500-700 rpm at about 100° C. under vacuum, wherein the reaction temperature is kept at 100° C. for 3-5 hours, controlling the quality of the resulting product by water dissolution and transparency measurement, where if transparency is not reached then the heating and transparency measurement continues every 30 minutes until transparancy is observed to quench the reation, with the temperature decreased for 30-60 minutes to 40-60° C.; emulsifying the entire mixture for 30 minutes at 400-800 rpm;
(v) filtering the product by injection through a nanofilter system before filling-packing.
